(19) United States
(12) Patent Application Publication
(10) Pub. No.: US 2025/0176962 A1
(43) Pub. Date: Jun. 5, 2025

(54) Sutureless Anastomosis Device and Uses Thereof

(71) Applicant: Washington University, St. Louis, MO (US)

(72) Inventors: Mohamed Zayed, St. Louis, MO (US); John Cashin, St. Louis, MO (US)

(73) Assignee: Washington University, St. Louis, MO (US)

(21) Appl. No.: 18/966,113

(22) Filed: Dec. 2, 2024

Related U.S. Application Data
(60) Provisional application No. 63/604,895, filed on Dec. 1, 2023.

(51) Int. Cl. A61B 17/11; A61B 17/00

(52) U.S. Cl. CPC A61B 17/11; A61B 2017/00862; A61B 2017/1107; A61B 2017/1135

(57) ABSTRACT

A sutureless anastomosis device configured to facilitate an anastomosis to a side of a damaged vessel is disclosed. The device includes inner and outer mounting faces configured to attached to the inner and outer vessel walls of the damaged vessel to anchor the device to the damaged vessel. The device further includes an outer cannula attached to the exposed surface of the outer mounting face; the outer cannula is configured to attach to a second vessel to form the side-to end anastomosis.

### CROSS-REFERENCE TO RELATED APPLICATIONS

This application claims the benefit of priority to U.S. Provisional Application Ser. No. 63/604,895 filed on Dec. 1, 2023, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE INVENTION

The present disclosure generally relates to a sutureless anastomosis device and uses thereof.

### BACKGROUND OF THE INVENTION

Traditional anastomosis suture-based end-to-side and end-to-end surgical techniques require added time and effort, as well as high technical skill. Alternative strategies that simplify the process could greatly improve clinical practices.

### SUMMARY OF THE INVENTION

Among the various aspects of the present disclosure is the provision of a sutureless anastomosis device and uses thereof.

In one aspect, a sutureless anastomosis device configured to facilitate an anastomosis to a side of a damaged vessel is disclosed. The device includes an inner mounting face comprising an inner contact surface, the inner contact surface configured to attach to an inner vessel wall of the damaged vessel; an outer mounting face comprising an outer contact surface, the outer contact surface configured to attach to an outer vessel wall of the damaged vessel; and an outer cannula attached at a first end to an exposed surface of the outer mounting face opposite the outer contact surface, the outer cannula configured to attach to a second vessel at a second end opposite to the first end. The inner and outer mounting faces are configured to compress a portion of a vessel membrane of the damaged vessel between the inner and outer contact surfaces; and the inner mounting face, the outer mounting face, and the outer cannula together define a cannula lumen forming a fluidic channel extending from a lumen of the damaged vessel, through the inner mounting face, the vessel wall, the outer mounting face, and the outer cannula to the second vessel. In some aspects, the device is configured to be deployed by catheter, wherein the inner and outer mounting faces are constructed from a flexible material configured to compress into a compressed configuration within a catheterized sheath and further configured to expand into an expanded configuration when deployed from the catheterized sheath, the expanded configuration comprising the inner and outer mounting faces compressing a portion of the vessel membrane of the damaged vessel between the inner and outer contact surfaces.

In some aspects, the inner and outer mounting faces are configured to conform to the curvature of the inner and outer vessel surfaces of the damaged blood vessel, respectively.

In some aspects, the outer mounting face further comprises a plurality of holes to facilitate suture placement and to enhance both axial and radial fixation of the outer mounting face to the vessel wall.

In some aspects, at least one of the inner and outer contact surfaces of the inner and outer mounting faces further comprise at least one vessel adhesion feature comprising a plurality of Velcro-like hooks, an adhesive compound, one or more holes configured to receive suture, and any combination thereof.

In some aspects, the inner mounting face further comprises an inner exposed face opposite the inner contact surface, the inner exposed face defining a cannula inner opening, wherein the cannula inner opening is configured to inhibit at least one of turbulence, steal, and vorticity induced by the deployment of the device into the damaged vessel.

In some aspects, the second vessel attached to the outer cannula is selected from a graft, a reservoir, and a blood vessel.

In some aspects, the outer cannula is configured to divert arterial or venous blood flow associated with the damaged vessel.

In some aspects, the inner and outer mounting faces are elliptically shaped.

In some aspects, the outer cannula further comprises at least one fixation feature on an outer surface of the outer cannula configured to enhance the strength of attachment of the second vessel to the outer cannula, wherein the at least one fixation feature is selected from a plurality of circumferential ridges, a plurality of Velcro-like hooks, an adhesive, at least one hole configured to receive a suture, and any combination thereof.

In another aspect, a method of repairing a damaged vessel is disclosed that includes performing an anastomosis to a side of the damaged vessel. The anastomosis is performed by providing a sutureless anastomosis device comprising an inner mounting face configured to attach to an inner vessel wall of the damaged vessel, an outer mounting face configured to attach to an outer vessel wall of the damaged vessel; and an outer cannula attached to the outer mounting face and configured to attach to a second vessel at a second end opposite to an attached end; compressing the outer and inner mounting faces into a compressed configuration within a catheterized sheath; inserting a catheter through a vessel wall of the damaged vessel onto the lumen of the catheter; advancing the compressed device within the catheterized sheath distally through the catheter until the compressed inner mounting face is positioned in the lumen of the damaged vessel and the compressed outer mounting face is positioned outside of the damaged vessel; withdrawing the catheter and catheterized sheath from the lumen of the damaged vessel to deploy the inner mounting face in an expanded configuration; withdrawing the catheter further to press the deployed inner mounting face against an inner vessel surface of the damaged vessel; and withdrawing the catheter and catheterized sheath completely to deploy the outer mounting face in the expanded configuration, wherein the deployed outer mounting face presses against the outer vessel surface of the damaged vessel.

In some aspects, the method further comprises attaching a second vessel to a protruding end of the outer cannula opposite the attached end to complete the anastomosis.

In some aspects, the method further comprises securing the outer mounting plate to the outer vessel surface of the damaged vessel by suturing through a plurality of holes defined in the outer mounting plate into the vessel wall of the damaged vessel.

In some aspects, the method further comprises securing the second vessel to the protruding end of the outer cannula by suturing through a plurality of holes defined in the protruding end of the outer cannula into the second vessel.

In some aspects, the second vessel is selected from a graft, a reservoir, or another vessel.

In some aspects, the damaged vessel and second vessel are vascular vessels, and the anastomosis is a vascular bypass graft selected from a coronary artery bypass, a mesenteric arterial bypass, a peripheral arterial bypass, or a venous vessel bypass.

In some aspects, the damaged vessel and second vessel are vascular vessels, and the anastomosis comprises a comprising an arteriovenous graft and a fistula.

In some aspects, the damaged vessel is a vascular vessel, and the second vessel comprises an element of a blood sampling device selected from a port, a central venous catheter, a vessel-associated reservoir.

### DESCRIPTION OF THE DRAWINGS

Those of skill in the art will understand that the drawings, described below, are for illustrative purposes only. The drawings are not intended to limit the scope of the present teachings in any way.

FIG. 1A is a side view drawing of a sutureless anastomosis device in accordance with one aspect of the present disclosure.

FIG. 1B is a cross-sectional side view drawing of a sutureless anastomosis device in accordance with one aspect of the present disclosure.

FIG. 1C is the cross-sectional side view drawing of FIG. 1B with a second vessel attached to a fixation feature of the sutureless anastomosis device in accordance with one aspect of the present disclosure.

FIG. 2A is a cross-sectional end of a sutureless anastomosis device in accordance with one aspect of the present disclosure.

FIG. 2B is a bottom-perspective view of the sutureless anastomosis device in accordance with one aspect of the present disclosure.

FIG. 3A contains side, cross-sectional, and top views of the sutureless anastomosis device in accordance with one aspect of the present disclosure.

FIG. 3B contains side, cross-sectional, and top views of the sutureless anastomosis device in accordance with one aspect of the present disclosure.

FIG. 3C contains side, cross-sectional, and top views of the sutureless anastomosis device in accordance with one aspect of the present disclosure.

FIG. 4A contains perspective and side views of a symmetric occluder used to occlude cardiac septal defects.

FIG. 4B contains perspective and side views of an eccentric occluder used to occlude cardiac septal defects.

FIG. 4C contains perspective and side views of a thin-waist occluder used to occlude cardiac septal defects.

FIG. 4D contains perspective and side views of an Amplatzer duct occluder used to occlude cardiac septal defects.

FIG. 5A is a schematic diagram illustrating a cardiac septal defect to be treated using an existing occluder; the method of inserting and expanding the occluder is provided as an analogous example of the insertion and expansion of the sutureless anastomosis device.

FIG. 5B is a schematic diagram showing an insertion of a catheter containing an existing occluder in a collapsed configuration through the cardiac septal defect of FIG. 5A, and the the expansion of a distal face of the occluder.

FIG. 5C is a schematic diagram showing the expansion of a proximal face of the occluder of FIG. 5B.

FIG. 5D is a schematic diagram showing the implanted occluder of FIG. 5C.

FIG. 6A contains a side view drawing of a sutureless anastomosis device in accordance with one aspect of the present disclosure.

FIG. 6B contains a close-up view of the outer cannula of the sutureless anastomosis device of FIG. 6A.

FIG. 7A is a schematic diagram showing the sutureless anastomosis device compressed within a catheterized sheath used to deploy the sutureless anastomosis device of FIG. 6A into a vessel.

FIG. 7B contains a schematic diagram showing the withdrawal of the catheterized sheath and deployment catheter and the associated expansion of the inner mounting surface of the device to prevent its withdrawal through the vasculature.

FIG. 7C contains a schematic diagram showing the withdrawal of the catheterized sheath and deployment catheter and associated expansion of the outer mounting surface of the device to anchor the device to the vessel wall.

### DETAILED DESCRIPTION OF THE INVENTION

The present disclosure is based, at least in part, on the discovery that a sutureless anastomosis device can be designed and employed to improve clinical practices. As shown herein, a sutureless anastomosis device for use in anastomosis surgery is disclosed.

In various aspects, the disclosed device provides for sutureless end-to-side arterial anastomosis, as opposed to traditional suture-based end-to-side and end-to-end surgical anastomoses. The disclosed device overcomes at least several shortcomings of existing surgical anastomosis devices and methods. Existing suture-based traditional anastomosis techniques require significant time and effort, as well as high technical skill to accomplish a stable vessel repair. The disclosed device and methods of use greatly simplifies the surgical process, as describe in additional detail below.

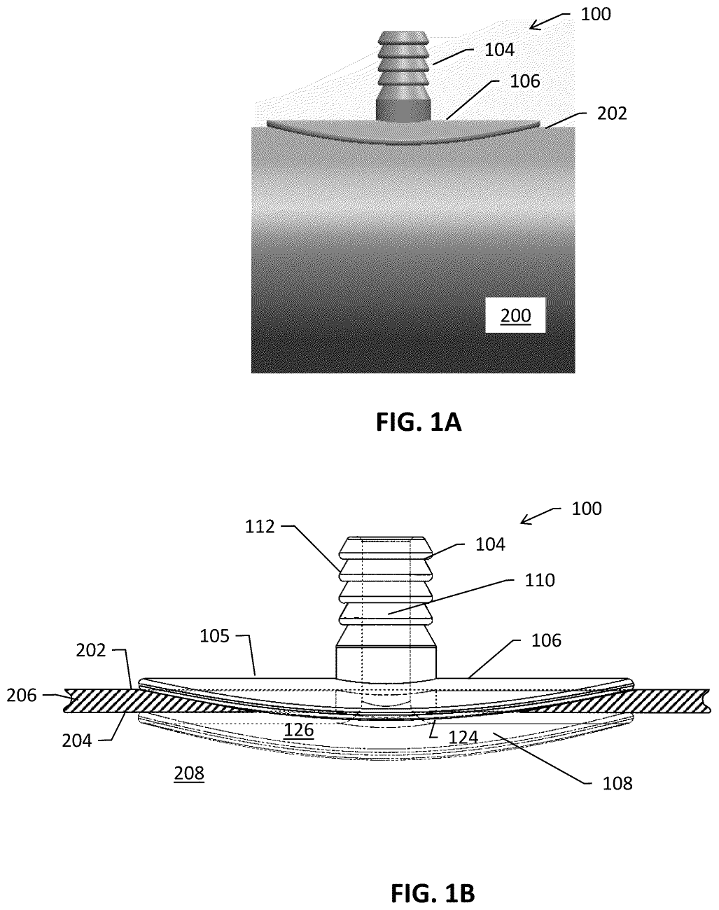

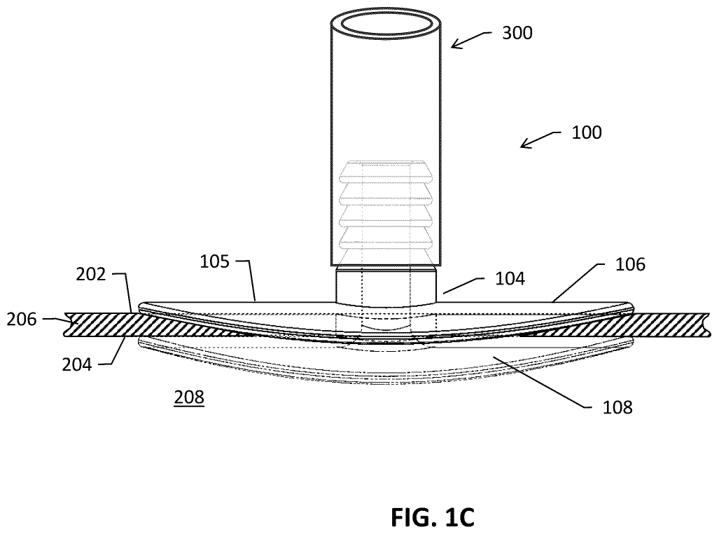

A sutureless anastomosis device 100 in one aspect is shown illustrated in FIGS. 1A and 1B. The device 100 is configured to deployed into a vessel 200 including, but not limited to, an artery via an arterial puncture or small arteriotomy. The device 100 may be introduced through the vessel wall 206 through a puncture through outer vessel surface 202 into the vessel lumen 208. The device 100 includes an outer mounting face 106 and inner mounting face 108 positioned against the outer vessel surface 202 and inner vessel surface 204, respectively. The outer mounting face 106 and inner mounting face 108 are configured to secure the device 100 in place by compressing against a segment of the vessel wall 206 positioned between the outer mounting face 106 and inner mounting face 108.

Referring again to FIGS. 1A and 1B, the device 100 further includes an outer cannula 104 attached to and projecting outward from an exposed surface 105 of the outer mounting face 106. As illustrated in FIG. 1B, the outer cannula 104 defines a lumen 110 configured to provide a fluidic conduit between the vessel lumen 208 and the outside of the vessel 200. In some aspects, the lumen 110 of the outer cannula 104 may have a constant diameter throughout. In other aspects, the lumen 110 of the outer cannula 104 may be inwardly or outwardly tapered, wherein the lumen diameter at the inner mounting face 108 is larger or smaller relative to the lumen diameter the outwardly protruding end of the outer cannula 104.

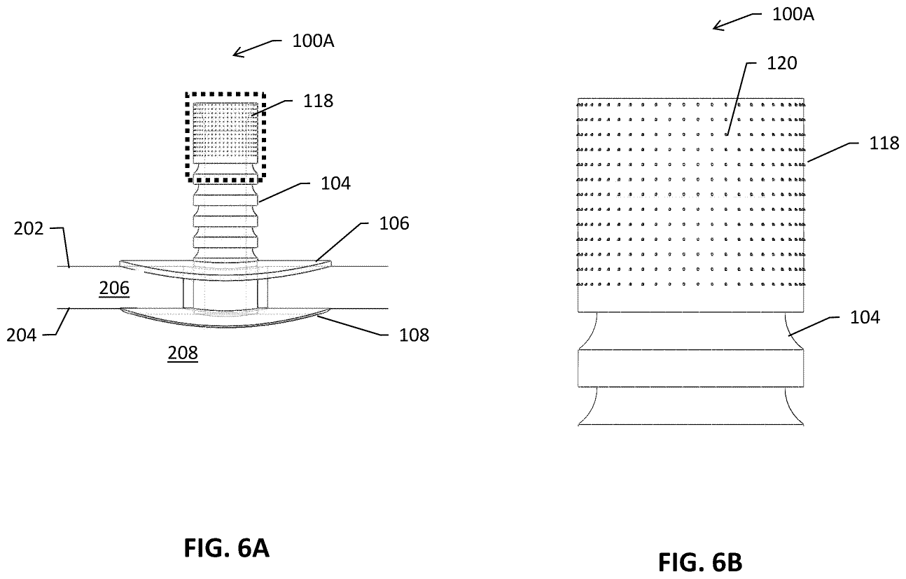

In various aspects, the outer cannula 104 is provided with at least one fixation feature 112 to facilitate the attachment of an end of a second vessel 300 to the outer cannula 104 to form the end-to-side anastomosis and to ensure both axial and radial fixation as illustrated in FIG. 1C. The outer cannula 104 may be provided with any suitable fixation fixture without limitation. Non-limiting examples of suitable fixation features include circumferential ridges as illustrated in FIG. 1B, an array of barbs as illustrated in FIGS. 6A and 6B, an adhesive, one or more holes configured to receive sutures, and/or any other suitable fixation feature without limitation.

In various aspects, the outer cannula 104 is configured for attachment to an additional element associated with an anastomosis procedure including, but not limited to a graft, a reservoir, or any other vessel without limitation. In some aspects, the outer cannula 104 provides for the diversion of arterial or venous blood flow. In other aspects, the disclosed device 100 is configured to implement a variety of other anastomosis procedures including, but not limited to, vascular bypass grafts such as those used for coronary artery bypass, mesenteric arterial bypass, peripheral arterial bypass, and venous vessel bypass; treatment of arteriovenous grafts or fistulas for hemodialysis; blood sampling such as blood sampling using ports, central venous catheters, and other vessel associated reservoirs. In various other aspects, the disclosed device 100 is configured for use in other anastomosis procedures including, but not limited to anastomosis of digestive tract vessels and any other suitable anastomosis procedure without limitation.

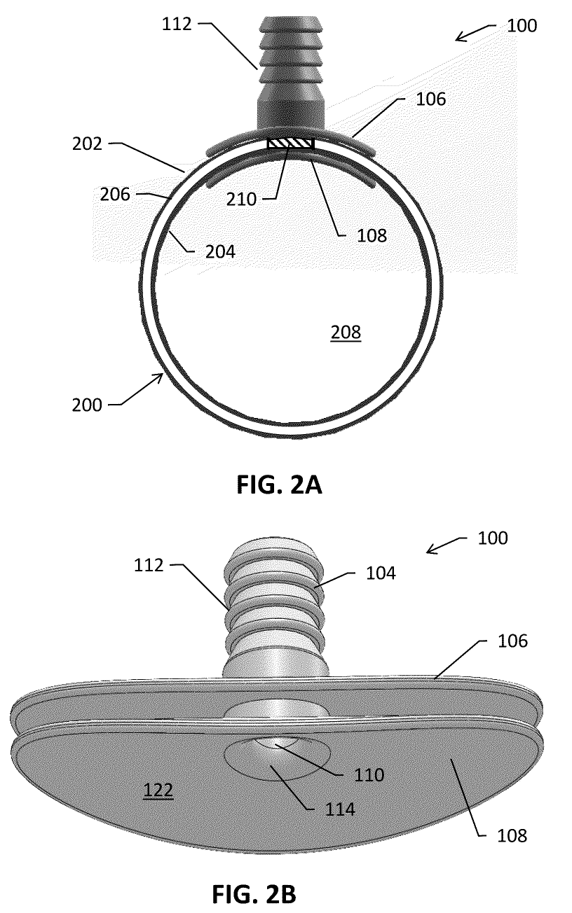

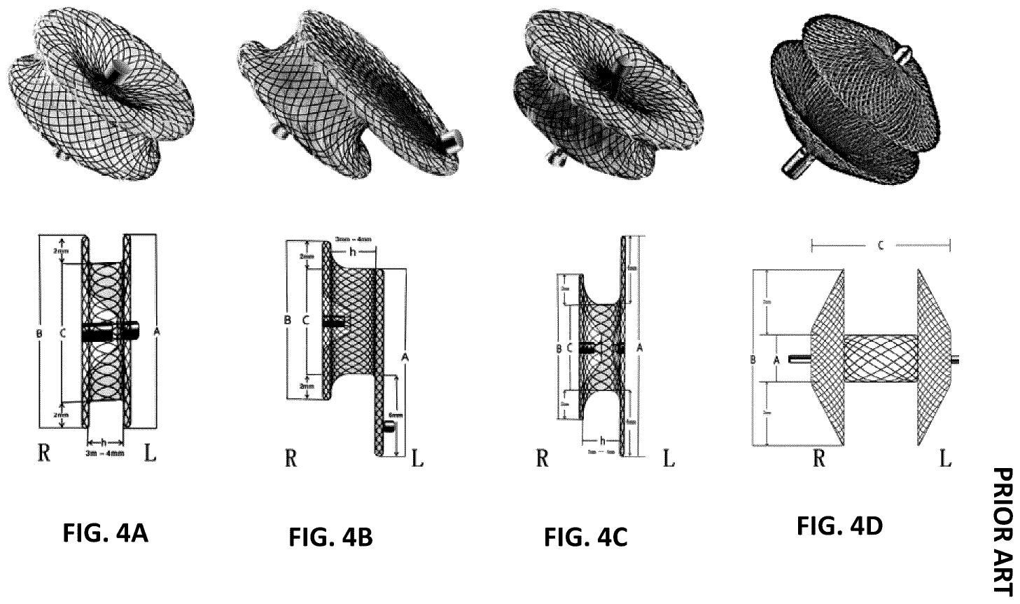

FIG. 2A is a cross-sectional view showing the device 100 deployed in the vessel 200 as described above. In some aspects, the outer and inner mounting faces 106/108 include a curvature matched to the contours of the outer and inner vessel surfaces 202/204, respectively to enhance the sealing of the puncture 210 through the vessel wall 206. In various aspects, the sizes and shapes of the outer and inner mounting faces 106/108 may be configured to minimize the area of application while ensuring a secure connection of the device 100 to the vessel wall 206. In various aspects, the sizes and shapes of the outer and inner mounting faces 106/108 may be relatively matched to one another. In other aspects, the sizes and/or shapes of the outer and inner mounting faces 106/108 may be different from one another. In various aspects, outer and inner mounting faces 106/108 may have shapes independently selected elliptical, circular, irregular, or any other suitable shape without limitation. Non-limiting examples of suitable sizes and shapes of the outer and inner mounting faces 106/108 are shown in FIGS. 4A, 4B, 4C, and 4D.

In various aspects, the shape and dimensions of various components of the device 100 may be adjusted, including but not limited to the length and circumference of the outer cannula 104, the diameter of the inner and outer openings of the outer cannula 104, the shapes and diameter(s) of the outer and inner mounting faces 106/108, and the length and spacing of fixation features 112 of the outer cannula 104.

FIG. 2B is a bottom perspective of the device 100 with the vessel 200 removed to enhance visualization. In various aspects, the exposed surface 122 of the inner mounting face 108 defines an inner portion of the lumen 110 of the outer cannula 104. In various aspects, the exposed surface 122 may further define a cannula inner opening 114. In some aspects, the cannula inner opening may be sized and shaped to facilitate flow through the lumen 110 of the outer cannula 104, and/or to minimize potential turbulence, steal, and/or vorticity.

In various aspects, the outer and inner mounting faces 106/108 may be formed of a flexible material or sheet. In some aspects, outer and inner mounting faces 106/108 of the device 100 may be configured to assume a flexed configuration to charactered by a reduced diameter to facilitate deployment of the device 100 into a vessel, as described in additional detail below. In some aspects, the outer and inner mounting faces 106/108 of the device 100 are configured to transition from the flexed configuration to a deployed configuration characterized by the full diameter and/or curvature to anchor the deployed device 100 in place as illustrated in FIGS. 1B and 2A.

In various aspects, the outer contact surface 126 and inner contact surface of the outer and inner mounting faces 106/108 of the device 100, respectively (see FIG. 1B) may further comprise one or more vessel adhesion features (not illustrated) to enhance adhesion to the outer and inner vessel surfaces, respectively. Non-limiting examples of vessel adhesion features include Velcro-like hooks, adhesive compounds, one or more holes configured to receive suture(s), and any other suitable vessel adhesion feature without limitation.

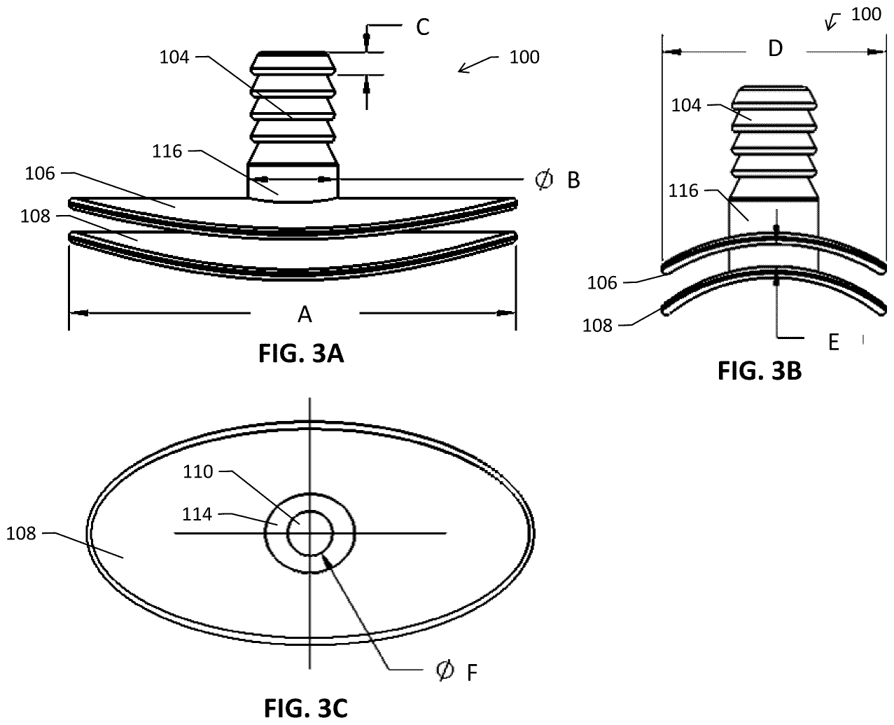

FIGS. 3A, 3B, and 3C are front, side, and bottom views of the device in one aspect. In this aspect, the outer mounting face 106 and inner mounting faces 108 may have matched elliptical shapes characterized by a major axis length A of about 10 mm and a minor axis length of about 5 mm. The outer cannula ridges may be 0.50 mm in length C. The inner opening of the outer cannula lumen 110 may be 1 mm and the outer opening may be 2 mm.

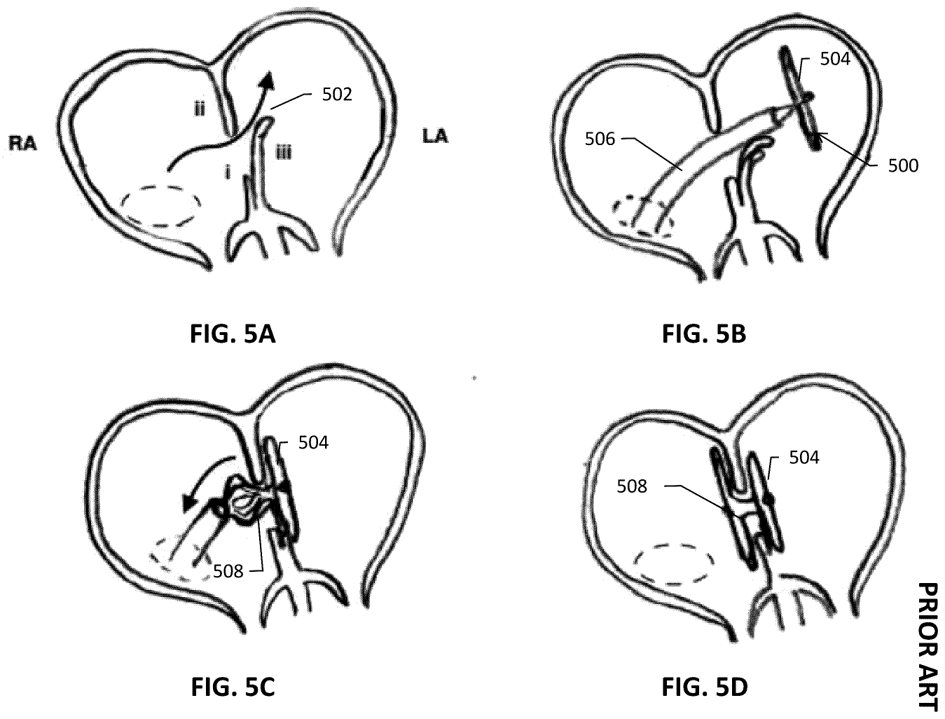

Without being limited to any particular theory, the disclosed device 100 may be deployed in a manner analogous to the deployment of existing cardiac septal/foramen occluders, as illustrated in FIGS. 5A, 5B, 5C, and 5D. The illustrated procedure is used to treat holes in the heart's septum 502 (FIG. 5A). An occluder 500 is delivered through a catheter 506 inserted through the hole 502, and the distal portion 504 of the occluder 500 may partially deployed to facilitate anchoring of the device 500 in the lumen (FIG. 5B). Once in place, the inner face of the distal portion 504 can be pulled gently back against the inner lumen of the septal wall, which allows the device 500 to conform to the curvature in the inner lumen wall (FIG. 5C), and the proximal portion 508 of the device 500 may be delivered in a compressed configuration. The proximal portion 508 of the device can then be deployed to facilitate placement of the outer mounting face, which locks and fixates the device in place, and outer mounting faces to better conform to the physiological structure of the vessel (FIG. 5D).

The disclosed device may be inserted percutaneously, wherein insertion of the device is done via needle-puncture through the skin. The disclosed device may be bound by a catheterized sheath, advanced perpendicularly through the vasculature, and deployed through the retraction of the sheath. This allows for the expansion of the inner-vasculature portion of the graft, preventing its withdrawal through the vasculature, followed by the expansion of the outer-vasculature anchoring section, anchoring the disclosed device in place (FIG. 6C, FIG. 6D, FIG. 6E).

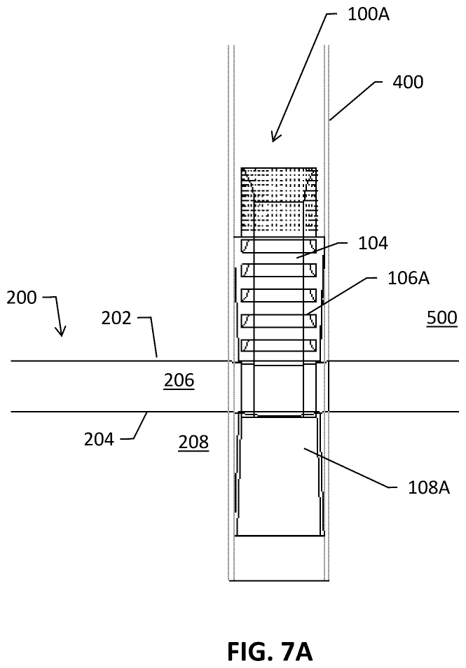

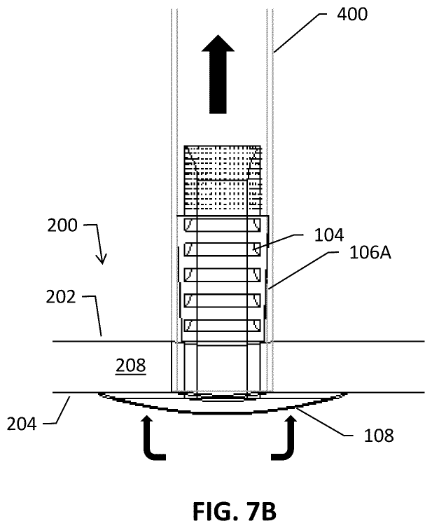

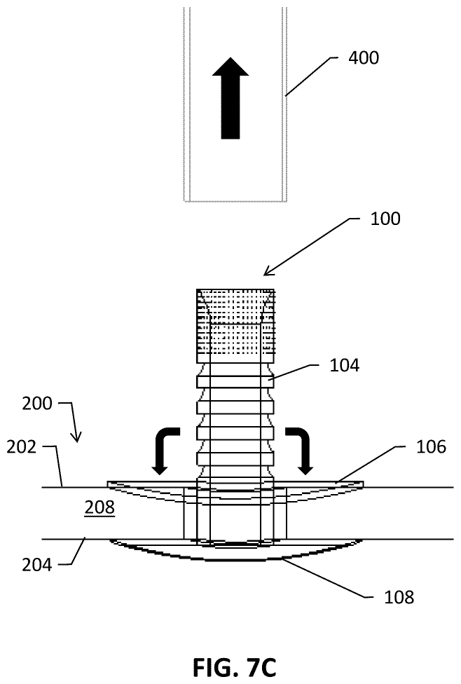

The deployment of the device 100 as part of an end-to-side anastomosis procedure using the device 100 of FIGS. 6A and 6B is illustrated schematically in FIGS. 7A, 7B, and 7C. FIG. 7A is cross-sectional view of a catheter 400 containing the compressed device 100A bound within a catheterized sheath (not illustrated). The catheter 400 is shown inserted through the vessel wall 206, and the compressed device 100 is positioned with the compressed inner mounting face 108A positioned within the vessel lumen 208 and the compressed outer mounting face 106A positioned in the external space 500 outside the vessel 200.

FIG. 7B is cross-sectional view showing the catheter 400 partially withdrawn from the vessel lumen 208 such that the distal end of the catheter 400 is positioned within the vessel wall 208. Upon the partial withdrawal of the catheter 400, the compressed inner mounting face 108A shown in FIG. 7A is released from the catheterized sheath to deploy the inner mounting face 108. The deployed inner mounting face 108 is pulled by the catheter 400 against the inner vessel surface 204 to anchor the device 100, as illustrated by the overlaid arrows.

FIG. 7C shows the device 100 after the catheter 400 is fully withdrawn from the vessel 200. Release of the remaining portions of the device 100 from the catheterized sheath cause the compressed outer mounting face 106A to deploy into the final deployed outer mounting face 106. The unfolding of the outer mounting face 106 further pulls the outer mounting face 106 closer toward the outer vessel surface 202, further securing the device 100 to the vessel wall 208.

### Therapeutic Methods

Also provided is a method of treating, preventing, or reversing a vessel-related disease, disorder, or symptom in a subject in need by repairing the vessel. In various aspects, the method includes performing an end-to-side anastomosis procedure to repair a vessel involved in the vessel-related disease, disorder, or symptom. In some aspects, the vessel-related disease, disorder, or symptom comprises a vascular disorder.

Methods described herein are generally performed on a subject in need thereof. A subject in need of the therapeutic methods described herein can be a subject having, diagnosed with, suspected of having, or at risk for developing a vessel-related disease. A determination of the need for treatment will typically be assessed by a history, physical exam, or diagnostic tests consistent with the disease or condition at issue. Diagnosis of the various conditions treatable by the methods described herein is within the skill of the art. Non-limiting examples of suitable subjects in need include an animal subject such as a mammal including, but not limited to horses, cows, dogs, cats, sheep, pigs, mice, rats, monkeys, hamsters, guinea pigs, and humans, or a bird subject including, but not limited to a chicken, duck, and turkey. In one non-limiting example, the subject is a human subject.

Generally, a safe and effective surgical intervention is, for example, an intervention that would cause the desired therapeutic effect in a subject while minimizing undesired side effects. In various embodiments, an effective surgical intervention described herein can substantially inhibit a vessel-related disease, slow the progress of a vessel-related disease, or limit the development of vessel-related disease.

The specific therapeutic surgical intervention for any particular subject will depend upon a variety of factors including the disorder being treated and the severity of the disorder; the age, body weight, general health, sex and diet of the subject; the location of the intervention; drugs used in combination or coincidental with the surgical intervention employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503).

Each of the states, diseases, disorders, and conditions, described herein, as well as others, can benefit from the devices and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Treatment in accordance with the methods described herein can be performed prior to, concurrent with, or after conventional treatment modalities for vessel-related diseases.

A surgical intervention can be performed simultaneously or sequentially with the administration of an agent, such as an antibiotic, an anti-inflammatory, or another agent. For example, a surgical intervention can be administered simultaneously with another agent, such as an antibiotic or an anti-inflammatory.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to the disclosed device components and a sterile container. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

What is claimed is:

1. A sutureless anastomosis device configured to facilitate an anastomosis to a side of a damaged vessel, the device comprising:
an inner mounting face comprising an inner contact surface, the inner contact surface configured to attach to an inner vessel wall of the damaged vessel;
an outer mounting face comprising an outer contact surface, the outer contact surface configured to attach to an outer vessel wall of the damaged vessel; and
an outer cannula attached at a first end to an exposed surface of the outer mounting face opposite the outer contact surface, the outer cannula configured to attach to a second vessel at a second end opposite to the first end;
wherein:
the inner and outer mounting faces are configured to compress a portion of a vessel membrane of the damaged vessel between the inner and outer contact surfaces; and
the inner mounting face, the outer mounting face, and the outer cannula together define a cannula lumen forming a fluidic channel extending from a lumen of the damaged vessel, through the inner mounting face, the vessel wall, the outer mounting face, and the outer cannula to the second vessel.

2. The device of claim 1, wherein the device is configured to be deployed by catheter, wherein the inner and outer mounting faces are constructed from a flexible material configured to compress into a compressed configuration within a catheterized sheath and further configured to expand into an expanded configuration when deployed from the catheterized sheath, the expanded configuration comprising the inner and outer mounting faces compressing a portion of the vessel membrane of the damaged vessel between the inner and outer contact surfaces.

3. The device of claim 1, wherein the inner and outer mounting faces are configured to conform to the curvature of the inner and outer vessel surfaces of the damaged blood vessel, respectively.

4. The device of claim 1, wherein the outer mounting face further comprises a plurality of holes to facilitate suture placement and to enhance both axial and radial fixation of the outer mounting face to the vessel wall.

5. The device of claim 1, wherein at least one of the inner and outer contact surfaces of the inner and outer mounting faces further comprise at least one vessel adhesion feature comprising a plurality of Velcro-like hooks, an adhesive compound, one or more holes configured to receive suture, and any combination thereof.

6. The device of claim 1, wherein the inner mounting face further comprises an inner exposed face opposite the inner contact surface, the inner exposed face defining a cannula inner opening, wherein the cannula inner opening is configured to inhibit at least one of turbulence, steal, and vorticity induced by the deployment of the device into the damaged vessel.

7. The device of claim 1, wherein the second vessel attached to the outer cannula is selected from a graft, a reservoir, and a blood vessel.

8. The device of claim 1, wherein the outer cannula is configured to divert arterial or venous blood flow associated with the damaged vessel.

9. The device of claim 1, wherein the inner and outer mounting faces are elliptically shaped.

10. The device of claim 7, wherein the outer cannula further comprises at least one fixation feature on an outer surface of the outer cannula configured to enhance the strength of attachment of the second vessel to the outer cannula, wherein the at least one fixation feature is selected from a plurality of circumferential ridges, a plurality of Velcro-like hooks, an adhesive, at least one hole configured to receive a suture, and any combination thereof.

11. A method of repairing a damaged vessel, the method comprising performing an anastomosis to a side of the damaged vessel, wherein the anastomosis is performed by:
providing a sutureless anastomosis device comprising an inner mounting face configured to attach to an inner vessel wall of the damaged vessel, an outer mounting face configured to attach to an outer vessel wall of the damaged vessel; and an outer cannula attached to the outer mounting face and configured to attach to a second vessel at a second end opposite to an attached end;
compressing the outer and inner mounting faces into a compressed configuration within a catheterized sheath;
inserting a catheter through a vessel wall of the damaged vessel onto the lumen of the catheter;
advancing the compressed device within the catheterized sheath distally through the catheter until the compressed inner mounting face is positioned in the lumen of the damaged vessel and the compressed outer mounting face is positioned outside of the damaged vessel;
withdrawing the catheter and catheterized sheath from the lumen of the damaged vessel to deploy the inner mounting face in an expanded configuration;
withdrawing the catheter further to press the deployed inner mounting face against an inner vessel surface of the damaged vessel; and
withdrawing the catheter and catheterized sheath completely to deploy the outer mounting face in the expanded configuration, wherein the deployed outer mounting face presses against the outer vessel surface of the damaged vessel.

12. The method of claim 11, further comprising attaching a second vessel to a protruding end of the outer cannula opposite the attached end to complete the anastomosis.

13. The method of claim 11, further comprising securing the outer mounting plate to the outer vessel surface of the damaged vessel by suturing through a plurality of holes defined in the outer mounting plate into the vessel wall of the damaged vessel.

14. The method of claim 11, further comprising securing the second vessel to the protruding end of the outer cannula by suturing through a plurality of holes defined in the protruding end of the outer cannula into the second vessel.

15. The method of claim 11, wherein the second vessel is selected from a graft, a reservoir, or another vessel.

16. The method of claim 11, wherein the damaged vessel and second vessel are vascular vessels, and the anastomosis is a vascular bypass graft selected from a coronary artery bypass, a mesenteric arterial bypass, a peripheral arterial bypass, or a venous vessel bypass.

17. The method of claim 11, wherein the damaged vessel and second vessel are vascular vessels, and the anastomosis comprises a comprising an arteriovenous graft and a fistula.

18. The method of claim 11, wherein the damaged vessel is a vascular vessels and the second vessel comprises an element of a blood sampling device selected from a port, a central venous catheter, a vessel-associated reservoir.
